(19) United States
(12) Patent Application Publication
(10) Pub. No.: US 2015/0238543 A1
(43) Pub. Date: Aug. 27, 2015

(54) COMPOSITIONS AND METHOD FOR TREATING NEUTRALIZING MICROORGANISMS

(71) Applicant: THE REGENTS OF THE UNIVERSITY OF MICHIGAN, Ann Arbor, MI (US)

(72) Inventors: Alex Rickard, Ann Arbor, MI (US); Jay Vornhagen, Seattle, WA (US); Lindsay Weir, Castleton, NY (US)

(21) Appl. No.: 14/422,086

(22) PCT Filed: Aug. 16, 2013
(86) PCT No.: PCT/US2013/055363

§ 371 Date: Feb. 17, 2015

Related U.S. Application Data
(60) Provisional application No. 61/684,567, filed on Aug. 17, 2012.

(51) Int. Cl. A61K 35/74; A01N 63/00

(52) U.S. Cl. CPC A61K 35/74; A01N 63/00

(57) ABSTRACT

The present disclosure relates to compositions and methods for targeted killing of microorganisms. In particular, the present disclosure relates to the use of Lysobacter gummosus and compositions containing Lysobacter gummosus in targeted killing of microorganisms in medical, industrial, domestic, or environmental applications, as well as treatment of bacterial infections (e.g., in biofilms).

This application claims priority to U.S. Provisional Patent Application No. 61/684,567, filed Aug. 17, 2012, which is herein incorporated by reference in its entirety.

### FIELD OF INVENTION

The present disclosure relates to compositions and methods for targeted killing of microorganisms. In particular, the present disclosure relates to the use of Lysobacter gummosus and compositions containing Lysobacter gummosus in targeted killing of microorganisms in medical, industrial, domestic, or environmental applications, as well as treatment of bacterial infections (e.g., in biofilms).

### BACKGROUND OF THE INVENTION

A biofilm is a well-organized community of microorganisms that adheres to surfaces and is embedded in the slimy extracellular polymeric substances (EPSs). EPSs are a complex mixture of high-molecular-mass polymers (>10,000 Da) generated by the bacterial cells, cell lysis and hydrolysis products, and organic matter adsorbed from the substrate. EPSs are involved in the establishment of stable arrangements of microorganisms in biofilms (Wolfaardt et al. (1998) Microb. Ecol. 35:213-223; herein incorporated by reference in its entirety), and extracellular DNA (eDNA) is one of the major components of EPSs (Flemming et al. (2001) Water Sci. Technol. 43:9-16; Spoering et al. (2006) Curr. Opin. Microbiol. 9:133-137; each herein incorporated by reference in its entirety). eDNA plays a very important role in biofilm development (Whitchurch et al. (2002) Science 295:1487; herein incorporated by reference in its entirety). It is involved in providing substrates for sibling cells, maintaining the three-dimensional structure of biofilms, and enhancing the exchange of genetic materials (Molin et al. (2003) Curr. Opin. Biotechnol. 13:255-261; Spoering et al. (2006) Curr. Opin. Microbiol. 9:133-137; each herein incorporated by reference in its entirety). Biofilm formation is one of the mechanisms bacteria use to survive in adverse environments (Costerton et al. (1995) Ann. Rev. Microbiol. 49:711-745; Hall-Stoodley et al. (2004) Nat. Rev. Microbiol. 2:95-108; O′Toole et al. (2000) Ann. Rev. Microbiol. 54:49-79; Parsek et al. (2003) Ann. Rev. Microbiol. 57:677-701; each herein incorporated by reference in its entirety). Bacteria living in a biofilm usually have significantly different properties from free-floating (planktonic) bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community. In some cases antibiotic resistance can be increased a thousand-fold (Stewart et al. (2001) Lancet 358:135-138; herein incorporated by reference in its entirety). Biofilms can be formed in various bacterial species (e.g., Acinetobacter sp. (e.g., A. baylyi, A. baumannii), Staphylococcus aureus, Stenotrophomonas maltophilia, Escherichia coli (e.g., E. coli K-12)). The formation of biofilms by such species is a major determinant of medical outcome during the course of colonization or infection. For example, Acinetobacter spp. frequently colonize patients in clinical settings through formation of biofilms on ventilator tubing, on skin and wound sites, medical tubing, and the like, and are a common cause of nosocomial pneumonia.

As biofilms are complex structures formed of various elements, their removal or disruption traditionally requires the use of dispersants, surfactants, detergents, enzyme formulations, antibiotics, biocides, boil-out procedures, corrosive chemicals, mechanical cleaning, use of antimicrobial agents, inhibiting microbial attachment, inhibiting biofilm growth by removing essential nutrients and promoting biomass detachment and degradation of biofilm matrix (Chen X S, P. S.: Biofilm removal caused by chemical treatments. Water Res 2000; 34:4229-4233; herein incorporated by reference in its entirety). However, such classical removal or disruption methods are not efficacious or feasible in all situations where biofilm formation is undesirable.

Additional methods for targeting pathogenic bacteria in biofilms are needed.

### SUMMARY OF THE INVENTION

The present disclosure relates to compositions and methods for targeted killing of microorganisms. In particular, the present disclosure relates to the use of Lysobacter gummosus and compositions containing Lysobacter gummosus in targeted killing of microorganisms in medical, industrial, domestic, or environmental applications, as well as treatment of bacterial infections (e.g., in biofilms).

For example, in some embodiments, the present invention provides compositions (e.g., pharmaceutical compositions), kits, formulations, etc. comprising Lysobacter gummosus and methods and uses of such compositions for killing or preventing the growth of microorganisms (e.g., bacteria).

For example, in some embodiments, the present invention provides a method of killing or preventing growth of microorganisms, comprising: contacting bacteria with a composition comprising Lysobacter gummosus, wherein said contacting kills or inhibits the growth of the microorganism. In some embodiments, bacteria are in a coaggregate or biofilm and the Lysobacter gummosus coaggregates with the bacteria. In some embodiments, microorganisms are on a surface of an object (e.g., a medical device, implantable medical device, etc.). In some embodiments, microorganisms are in or on a subject (e.g., in a wound). In some embodiments, microorganisms are in a coaggregate or biofilm with a plurality of different species. In some embodiments, Lysobacter gummosus kills a subset of microorganisms in the coaggregate or biofilm (e.g., Lysobacter gummosus selectively kills specific microorganism species but not others (e.g., including but not limited to, of Staphylococcys warneri, Streptococcus sp., Staphylococcus epidermidis, Blastomonas natatoria, Methylobacterium sp., Sphingomonas sp., Streptococcus gordonii, Staphylococcus aureus (e.g., antibiotic resistant isolates), Salmonella enterica subsp. Enterica, Candida albicans, Acinetobacter baumanii, Micrococcus luteus, Emticicia sp., Staphylococcus aureus, Chrysobacter sp., Staphylococcus capreae, Staphylococcus warneri, or Corynebacterium singular). In some embodiments, the Lysobacter gummosus is formulated as a pharmaceutical composition, a disinfecting or cleaning solution, or is on or in a wound dressing).

The present invention further provides uses of Lysobacter gummosus in the killing or prevention of growth of microorganisms (e.g., bacteria).

Additional embodiments of the present invention provide compositions and kits comprising isolated and/or purified Lysobacter gummosus. In some embodiments, the Lysobacter gummosus is lyophilized. In some embodiments, Lysobacter gummosus is genetically engineered to alter its virulence, specificity, or growth.

Additional embodiments are described herein.

### DESCRIPTION OF THE FIGURES

FIG. 1 shows a neighbor-joining phylogenetic tree of the partial 16S rRNA gene sequences from isolates cultured from the three showerhead biofilms.

FIG. 2 shows confocal Laser scanning microscope images showing the ability of two autoaggregating species to coaggregate. Cell suspensions of (A) M. hispanicum AH007 and, (B) M. trichothecenolyticum HM016, and (C) coaggregating autoaggregates of M. hispanicum AH007 and M. trichothecenolyticum HM016 are shown.

FIG. 3 shows confocal laser scanning microscope images demonstrating the typical interdigitated nature of coaggregation between three showerhead biofilm species that coaggregated with one another. Cell suspensions of (A) M. luteus AH004, (B) B. lenta HM006, (C) L. gummosus HM010, and coaggregates of (D) B. lenta HM006 and L. gummosus HM010 (visual score of 4), (E) M. luteus AH004 and B. lenta HM006 (visual score of 4), and (F) M. luteus AH004 and L. gummosus HM010 (visual score of 2).

FIG. 4 shows a diagrammatic representation of the inter- and intra-biofilm specificity of coaggregation between the showerhead biofilm isolates after growth in batch culture for 48h. The thickest ling represents a visual Coaggregation score of 4, line of intermediate thickness represents a score of 3, and thinnest dotted line represents a score of 2.

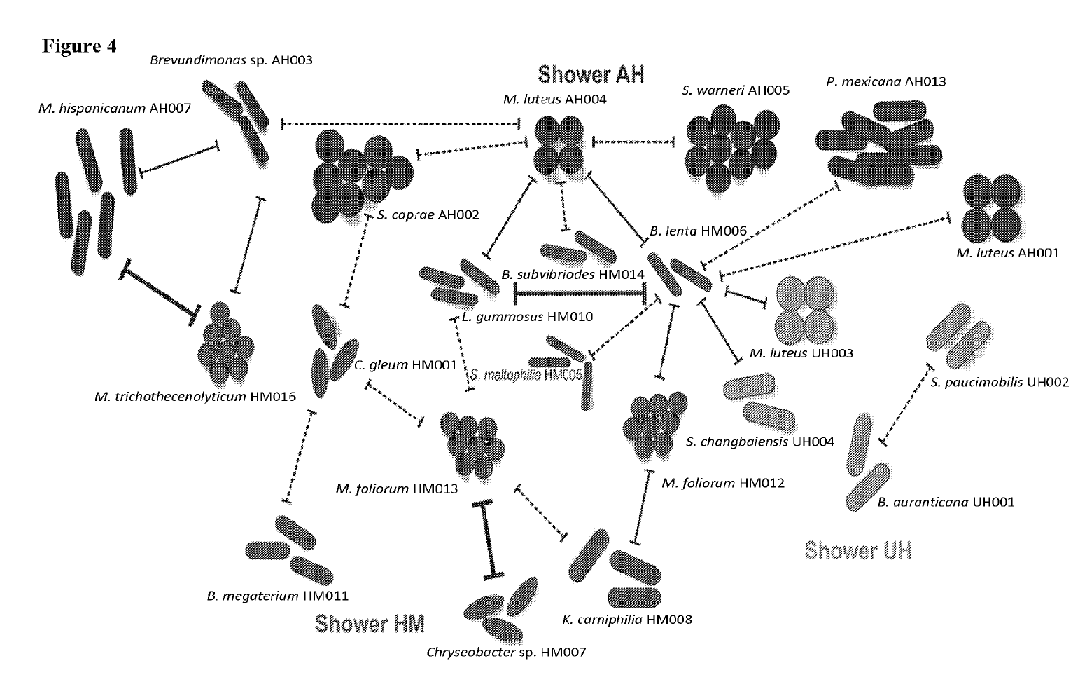

FIG. 5 shows camera images collected over an eleven day period showing the co-growth of Lysobacter gummosus HM010 and a clinically derived strain of Staphylococcus aureus (left-hand plate, strain 483, also known as CWS34 in Rickard et al. [Rickard et al., (2010). J Appl Microbiol. 108(5):1509-1522]) and a strain of Salmonella enterica subsp. enterica (right hand plate, strain ATCC 14028) on R2A agar plates at approximately 25° C. (A) Initial inoculation of plates at Time 0. (B) Co-growth of organisms after four days and killing of S. aureus 483 as indicated by thinning-out (lysing) of colony streaks that are close to L. gummosus HM010. (C) Spreading of L. gummosus HM010 across agar plates, after seven days incubation, and lysing of S. aureus 483 (as indicated by S. aureus colony disappearance). Limited lysing was observed for S. enterica ATCC14028. (D) Continued spreading of L. gummosus HM010 and extensive lysing of S. aureus 483. Some S. enterica ATCC14028.

FIG. 6 shows cell killing within a coaggregate.

### DEFINITIONS

To facilitate an understanding of the present invention, a number of terms and phrases are defined below:

As used herein the term “biofilm” refers to any three-dimensional, (e.g., matrix-encased) microbial community displaying multicellular characteristics. Accordingly, as used herein, the term biofilm includes surface-associated biofilms as well as biofilms in suspension, such as flocs and granules. Biofilms may comprise a single microbial species or may be mixed species complexes, and may include bacteria as well as fungi, algae, protozoa, or other microorganisms.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used herein, the term “subject” refers to individuals (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment for a condition characterized by the presence of biofilm-forming bacteria, or in anticipation of possible exposure to biofilm-forming bacteria.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “virulence” refers to the degree of pathogenicity of a microorganism (e.g., bacteria or fungus), e.g., as indicated by the severity of the disease produced or its ability to invade the tissues of a subject. It is generally measured experimentally by the median lethal dose (LD₅₀) or median infective dose (ID₅₀). The term may also be used to refer to the competence of any infectious agent to produce pathologic effects.

As used herein, the term “effective amount” refers to the amount of a composition (e.g., a composition comprising L. gummosus) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions comprising L. gummosus) to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.), topical administration and the like.

As used herein, the term “treating a surface” refers to the act of exposing a surface to one or more compositions comprising L. gummosus. Methods of treating a surface include, but are not limited to, spraying, misting, submerging, and coating.

As used herein, the term “co-administration” refers to the administration of at least two agent(s) (e.g., L. gummosus in combination with an antibiotic) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “wound” refers broadly to injuries to tissue including the skin, subcutaneous tissue, muscle, bone, and other structures initiated in different ways, for example, surgery, (e.g., open post cancer resection wounds, including but not limited to, removal of melanoma and breast cancer etc.), contained post-operative surgical wounds, pressure sores (e.g., from extended bed rest) and wounds induced by trauma. As used herein, the term “wound” is used without limitation to the cause of the wound, be it a physical cause such as bodily positioning as in bed sores or impact as with trauma or a biological cause such as disease process, aging process, obstetric process, or any other manner of biological process. Wounds caused by pressure may also be classified into one of four grades depending on the depth of the wound: i) Grade I: wounds limited to the epidermis; ii) Grade II: wounds extending into the dermis; iii) Grade III: wounds extending into the subcutaneous tissue; and iv) Grade IV: wounds wherein bones are exposed (e.g., a bony pressure point such as the greater trochanter or the sacrum). The term “partial thickness wound” refers to wounds that are limited to the epidermis and dermis; a wound of any etiology may be partial thickness. The term “full thickness wound” is meant to include wounds that extend through the dermis.

As used herein, “wound site” refers broadly to the anatomical location of a wound, without limitation.

As used herein, the term “dressing” refers broadly to any material applied to a wound for protection, absorbance, drainage, treatment, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) (Kannon and Garrett (1995) Dermatol. Surg. 21: 583-590; Davies (1983) Burns 10: 94; each herein incorporated by reference). The present invention also contemplates the use of dressings impregnated with pharmacological compounds (e.g., antibiotics, antiseptics, thrombin, analgesic compounds, etc). Cellular wound dressings include commercially available materials such as Apligraf®, Dermagraft®, Biobrane®, TransCyte®, Integra® Dermal Regeneration Template®, and OrCell®.

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., L. gummosus) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells which line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers, and adjuvants. (See e.g., Martin, Remington's Pharmaceutical Sciences, 15th Ed., Mack Publ. Co., Easton, Pa. (1975), incorporated herein by reference). In certain embodiments, the compositions of the present invention may be formulated for veterinary, horticultural or agricultural use. Such formulations include dips, sprays, seed dressings, stem injections, sprays, and mists. In certain embodiments, compositions of the present invention may be used in any application where it is desirable to alter (e.g., inhibit) the formation of biofilms, e.g., food industry applications; consumer goods (e.g., medical goods, goods intended for consumers with impaired or developing immune systems (e.g., infants, children, elderly, consumers suffering from disease or at risk from disease), and the like.

As used herein, the term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, contact lenses, and toothbrushes. Birth control devices include, but are not limited to, intrauterine devices (IUDs), diaphragms, and condoms.

As used herein, the term “therapeutic agent,” refers to compositions that decrease the infectivity, morbidity, or onset of mortality in a subject (e.g., a subjected contacted by a biofilm-forming microorganism) or that prevent infectivity, morbidity, or onset of mortality in a host contacted by a biofilm-forming microorganism. As used herein, therapeutic agents encompass agents used prophylactically, e.g., in the absence of a biofilm-forming organism, in view of possible future exposure to a biofilm-forming organism. Such agents may additionally comprise pharmaceutically acceptable compounds (e.g., adjuvants, excipients, stabilizers, diluents, and the like). In some embodiments, the therapeutic agents of the present invention are administered in the form of topical compositions, injectable compositions, ingestible compositions, and the like. When the route is topical, the form may be, for example, a solution, cream, ointment, salve or spray.

As used herein, the term “pathogen” refers to a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

As used herein, the term “microbe” refers to a microorganism and is intended to encompass both an individual organism, or a preparation comprising any number of the organisms.

As used herein, the term “microorganism” refers to any species or type of microorganism, including but not limited to, bacteria, archaea, fungi, protozoans, mycoplasma, and parasitic organisms.

As used herein, the term “fungi” is used in reference to eukaryotic organisms such as the molds and yeasts, including dimorphic fungi.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram-stain, causing the stained cells to generally appear dark blue to purple under the microscope. “Gram-negative bacteria” do not retain the primary dye used in the Gram-stain, but are stained by the counterstain. Thus, Gram-negative bacteria generally appear red.

The term “non-pathogenic bacteria” or “non-pathogenic bacterium” includes all known and unknown non-pathogenic bacterium (Gram-positive or Gram-negative) and any pathogenic bacterium that has been mutated or converted to a non-pathogenic bacterium. Furthermore, a skilled artisan recognizes that some bacteria may be pathogenic to specific species and non-pathogenic to other species; thus, these bacteria can be utilized in the species in which it is non-pathogenic or mutated so that it is non-pathogenic.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells, including, e.g., prokaryotic cells and eukaryotic cells. Included within this term are continuous cell lines (e.g., with an immortal phenotype), primary cell cultures, transformed cell lines, finite cell lines (e.g., non-transformed cells), bacterial cultures in or on solid or liquid media, and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

The term “coating” as used herein refers to a layer of material covering, e.g., a medical device or a portion thereof. A coating can be applied to the surface or impregnated within the material of the implant.

As used herein, the term “antimicrobial agent” refers to composition that decreases, prevents or inhibits the growth of bacterial and/or fungal organisms. Examples of antimicrobial agents include, e.g., antibiotics and antiseptics and L. gummosus.

The term “antiseptic” as used herein is defined as an antimicrobial substance that inhibits the action of microorganisms, including but not limited to a-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, silver, benzyl peroxide, alcohols, and carboxylic acids and salts. One skilled in the art is cognizant that these antiseptics can be used in combinations of two or more to obtain a synergistic or additive effect. Some examples of combinations of antiseptics include a mixture of chlorhexidine, chlorhexidine and chloroxylenol, chlorhexidine and methylisothiazolone, chlorhexidine and (α-terpineol, methylisothiazolone and α-terpineol; thymol and chloroxylenol; chlorhexidine and cetylpyridinium chloride; or chlorhexidine, methylisothiazolone and thymol. These combinations provide a broad spectrum of activity against a wide variety of organisms.

The term “antibiotics” as used herein is defined as a substance that inhibits the growth of microorganisms, preferably without damage to the host. For example, the antibiotic may inhibit cell wall synthesis, protein synthesis, nucleic acid synthesis, or alter cell membrane function.

Classes of antibiotics include, but are not limited to, macrolides (e.g., erythromycin), penicillins (e.g., nafcillin), cephalosporins (e.g., cefazolin), carbepenems (e.g., imipenem), monobactam (e.g., aztreonam), other beta-lactam antibiotics, beta-lactam inhibitors (e.g., sulbactam), oxalines (e.g. linezolid), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin), polyenes (e.g., amphotericin B), azoles (e.g., fluconazole), and echinocandins (e.g., caspofungin acetate).

Examples of specific antibiotics include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al, U.S. Pat. No. 4,642,104 herein incorporated by reference will readily suggest themselves to those of ordinary skill in the art.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present disclosure relates to compositions and methods for targeted killing of microorganisms. In particular, the present disclosure relates to the use of Lysobacter gummosus and compositions containing Lysobacter gummosus in targeted killing of microorganisms in medical, industrial, domestic, or environmental applications, as well as treatment of bacterial infections (e.g., in biofilms).

Coaggregation is the highly specific recognition and adhesion of genetically distinct bacteria mediated by complementary protein adhesins and polysaccharide receptors on the cell surface of coaggregating cells (Kolenbrander, Annu Rev Microbiol 54, 413-437, 2000; Rickard et al., Trends Microbiol 11, 94-100, 2003a). This phenomenon is distinct from autoaggregation, which is the recognition and adhesion of genetically identical bacteria to one another (Khemaleelakul et al., J Endod 32, 312-318, 2006; Rickard et al., FEMS Microbiol Lett 220, 133-140, 2003b; Van Houdt & Michiels, Res Microbiol 156, 626-633, 2005). Coaggregation was first described between human dental plaque bacteria in 1970 (Gibbons & Nygaard, Arch Oral Biol 15, 1397-1400, 1970), and work over the last two decades has shown that it also occurs between bacteria isolated from the human gut, the human urogenital tract, in wastewater flocs, and freshwater biofilms (Ledder et al., FEMS Microbiol Ecol 66, 630-636, 2008; Phuong et al., J Biotechnol, 2011; Reid et al., Can J Microbiol 34, 344-351, 1988; Rickard et al., Appl Environ Microbiol 66, 431-434, 2000; Simoes et al., Appl Environ Microbiol 74, 1259-1263, 2008). Coaggregation has also been shown to occur among numerous taxonomically distinct freshwater species (Rickard et al., Appl Environ Microbiol 68, 3644-3650, 2002; Rickard et al., 2003b, supra; Rickard et al., Appl Environ Microbiol 70, 7426-7435, 2004b; Simoes et al., Appl Environ Microbiol 74, 1259-1263, 2008) and in planktonic and biofilm populations (Rickard et al., J Appl Microbiol 96, 1367-1373, 2004a). Studies of coaggregation between Sphingomonas (Blastomonas) natatoria and Micrococcus luteus demonstrated that the ability of a species to coaggregate alters dual-species biofilm development in both flowing and static environments (Min & Rickard, Appl Environ Microbiol 75, 3987-3997, 2009; Min et al., Biofouling 26, 931-940, 2010). Coaggregation may mediate biofilm development, architectural changes, and species composition (Hojo et al., J Dent Res 88, 982-990, 2009; Kolenbrander et al., Periodontol 2000 42, 47-79, 2006; Rickard et al., 2003a, supra). In addition, coaggregation may play a role in promoting or hindering the integration of pathogenic species into freshwater biofilms (Buswell et al., Appl Environ Microbiol 64, 733-741, 1998). Evidence to support such a possibility can be found in studies of dental plaque biofilms where coaggregation has been indicated to promote the integration of oral pathogens such as Porphyromonas gingivalis (Kolenbrander et al., Periodontol 2000 42, 47-79, 2006; Whitmore & Lamont, Mol Microbiol 81, 305-314, 2011).

A biofilm is an aggregate of microorganisms in which cells adhere to each other and/or to a surface. These adherent cells are frequently embedded within a self-produced matrix of extracellular polymeric substance (EPS). Biofilm EPS, also referred to as slime, is a polymeric conglomeration generally composed of extracellular DNA, proteins, and polysaccharides in various configurations and of various compositions. Biofilms may form on living or non-living surfaces, and represent a prevalent mode of microbial life in natural, industrial and clinical settings. The microbial cells growing in a biofilm are physiologically distinct from planktonic cells of the same organism, which, by contrast, are single cells that may float or swim in a liquid medium.

Microbial biofilms form in response to many factors including but not limited to cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated (Petrova et al., J. Bacteriol. 2012 May; 194(10):2413-25; Stoodley et al., Annu Rev Microbiol. 2002; 56:187-209).

Although the present invention is not limited by any type of biofilm, biofilm formation typically begins with the attachment of free-floating microorganisms to a surface. These first colonists adhere to the surface initially through weak, reversible Van der Waals forces. If the colonists are not immediately separated from the surface, they can anchor themselves more permanently using cell adhesion structures such as pili.

Initial colonists commonly facilitate the arrival of other cells by providing more diverse adhesion sites and beginning to build the matrix that holds the biofilm together. Some species are not able to attach to a surface on their own but are often able to anchor themselves to the matrix or directly to earlier colonists. It is during this colonization that the cells are able to communicate via quorum sensing, for example, using such compounds as AHL. Once colonization initiates, the biofilm grows through a combination of cell division and recruitment. The final stage of biofilm formation is known as development although herein the terms “formation” and “development” are used interchangeably. In this final stage, the biofilm is established and may only change in shape and size. The development of a biofilm may allow for an aggregate cell colony (or colonies) to be increasingly antibiotic resistant.

Dispersal of cells from the biofilm colony is an essential stage of the biofilm lifecycle. Dispersal enables biofilms to spread and colonize new surfaces. Enzymes that degrade the biofilm extracellular matrix, such as dispersin B and deoxyribonuclease, may play a role in biofilm dispersal (Whitchurch et al. (2002) Science 295:1487; herein incorporated by reference in its entirety). Biofilm matrix degrading enzymes may be useful as anti-biofilm agents (Kaplan et al. (2004) Antimicrobial Agents and Chemotherapy 48 (7): 2633-6; Xavier et al. (2005) Microbiology 151 (Pt 12): 3817-32; each herein incorporated by reference in its entirety). A fatty acid messenger, cis-2-decenoic acid, can induce dispersion and inhibiting growth of biofilm colonies. Secreted by Pseudomonas aeruginosa, this compound induces dispersion in several species of bacteria and the yeast Candida albicans (Davies et al. (2009) Journal of Bacteriology 191 (5): 1393-403; herein incorporated by reference in its entirety).

Biofilms are ubiquitous and are usually found on solid substrates submerged in or exposed to some aqueous solution, although they can form as floating mats on liquid surfaces and also on the surface of leaves, particularly in high humidity climates. Given sufficient resources for growth, a biofilm will quickly grow to be macroscopic. Many types of microbes can form biofilms, e.g., bacteria, archaea, protozoa, fungi and algae. Biofilms may comprise a single type of microbe (monospecies biofilms), or, commonly, multiple types. In some mixed species biofilms, each group performs specialized metabolic functions.

Biofilms form in environments including but not limited to: substrates (e.g., rocks, pebbles) in natural bodies of water (e.g., rivers, pools, streams, oceans, springs); extreme environments (e.g., hot springs including waters with extremely acidic or extremely alkaline pH; frozen glaciers); residential and industrial settings in which solid surfaces are exposed to liquid (e.g., showers, water and sewage pipes, floors and counters in food preparation or processing areas, water-cooling systems, marine engineering systems); hulls and interiors of marine vessels; sewage and water treatment facilities (e.g., water filters, pipes, holding tanks); contaminated waters; within or upon living organisms (e.g., dental plaque, surface colonization or infection of e.g., skin, surfaces of tissues or organs or body cavities or at wound sites; plant epidermis, interior of plants); on the inert surfaces of implanted devices such as catheters, prosthetic cardiac valves, artificial joints, and intrauterine devices; and the like.

Biofilms are involved in a wide variety of microbial infections in the body. Infectious processes in which biofilms have been implicated include but are not limited to urinary tract infections, catheter infections, middle-ear infections, formation of dental plaque and gingivitis, contact lens contamination (Imamura et al. (2008) Antimicrobial Agents and Chemotherapy 52 (1): 171-82; herein incorporated by reference in its entirety), and less common but more lethal processes such as endocarditis, infections in cystic fibrosis, and infections of permanent indwelling devices such as joint prostheses and heart valves (Lewis et al. (2001) Antimicrobial Agents and Chemotherapy 45 (4): 999-1007; Parsek et al. (2003) Annual Review of Microbiology 57: 677-701; each herein incorporated by reference in its entirety). Bacterial biofilms may impair cutaneous wound healing and reduce topical antibacterial efficiency in healing or treating infected skin wounds (Davis et al. (2008) Wound Repair and Regeneration 16 (1): 23-9; herein incorporated by reference in its entirety).

Experiments conducted during the course of development of embodiments of the present invention identified and isolated a bacterial strain growing on the exposed surface of a domestic showerhead as part of a multi-species biofilm. The bacterium was identified by partial 16S rRNA gene sequencing as being Lysobacter gummosus. The bacterium was one of a collection of species that were shown to coaggregate with each other (from the same showerhead biofilm) and with others isolated from different sources. Subsequent analysis of the strain showed that it exhibits a potent lytic activity against numerous other bacteria. These include, but are not limited to, of Staphylococcys warneri, Staphylococcus epidermidis, Blastomonas natatoria, Methylobacterium sp., Sphingomonas sp., Streptococcus gordonii, Staphylococcus aureus 483, Salmonella enterica subsp. Enterica, Candida albicans, Acinetobacter baumanii, Micrococcus luteus, Emticicia sp., Staphylococcus aureus, Chrysobacter sp., Staphylococcus capreae, Staphylococcus warneri, Corynebacterium singular and a highly antibiotic resistant clinical isolate of Staphylococcus aureus. In addition, experiments demonstrated that L. gummosus demonstrated gliding motility. As such, on an agar plate, L. gummosus moves toward and destroys (lyses) the target species (e.g., using the components of the destroyed cells as an energy source to perpetuate killing).

Accordingly, embodiments of the present invention provide compositions (e.g., pharmaceutical or research compositions or kits) comprising L. gummosus and pharmaceutical, industrial, or research methods of using the bacteria in the treatment and prevention of bacterial infections and in decontamination of surfaces (e.g., surfaces of medical devices). In some embodiments, L. gummosus is isolated or purified (e.g., from growth media and/or other bacteria). In some embodiments, L. gummosus utilized in methods and compositions described herein is at least 50%, at least 75%, at least 80%, at least 85%, at least 90%, at least 95%, or at least 99% pure.

In some embodiments, L. gummosus bacteria are live cells or freeze-dried cells. In some embodiments, bacteria are lyophilized. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, mineral oil, glycerol, linseed oil. The viability of freeze-dried bacteria in oil is maintained for at least nine months. Optionally live cells can be added to one of the above oils and stored.

In some embodiments, two bacteria are spray-dried. In other embodiments, bacteria are suspended in an oil phase and are encased by at least one protective layer, which is water-soluble (water-soluble derivatives of cellulose or starch, gums or pectins; See e.g., EP 0 180 743, herein incorporated by reference in its entirety).

In some embodiments, the present invention provides compositions comprising one or more distinct isolated L. gummosus bacteria, alone or in combination with a pharmaceutically acceptable carrier or other desired delivery material (e.g., cleaner or disinfectant, etc.). Pharmaceutical compositions and formulations for topical administration may include transdermal patches, ointments, lotions, creams, gels, drops, suppositories, sprays, liquids, mouthwash, and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations, which may conveniently be presented in unit dosage form, may be prepared according to conventional techniques well known in the pharmaceutical industry.

The compositions may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions, such as dyes, flavoring agents, preservatives, antioxidants, opacifiers, thickening agents and stabilizers. However, such materials, when added, should not unduly interfere with the biological activities of the components of the compositions. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

In some embodiments, the pharmaceutical composition contains a) L. gummosus, and b) one or more other agents useful in killing or preventing the growth of microorganisms (e.g., antibiotics) or impacting the growth, formation or health impact or microorganisms in biofilms.

In some embodiments, the present invention provides kits, pharmaceutical compositions, or other delivery systems for use of L. gummosus in treating or preventing bacterial infections or biofilms present on surfaces. The kit may include any and all components necessary, useful or sufficient for research or therapeutic uses including, but not limited to, L. gummosus pharmaceutical carriers, and additional components useful, necessary or sufficient for treating or preventing bacterial infections. In some embodiments, the kits provide a sub-set of the required components, wherein it is expected that the user will supply the remaining components. In some embodiments, the kits comprise two or more separate containers wherein each container houses a subset of the components to be delivered. Optionally, compositions and kits comprise other active components in order to achieve desired therapeutic effects.

In some embodiments, L. gummosus is used to kill bacteria in coaggregates or biofilms. The present invention is not limited to a particular mechanism. Indeed, an understanding of the mechanism is not necessary to practice the present invention. Nonetheless, it is contemplated that by incorporating into coaggregates or biofilms, L. gummosus is able to increase killing efficiency via gliding and proximity.

In some embodiments, L. gummosus targets certain pathogens in a coaggragate or biofilm but not others. This provides the advantage of selective killing in situations where certain pathogens are to be targeted and killed but the other (non-sensitive) species are preferred to be left alive. In addition, in some embodiments, compositions comprising L. gummosus are utilized for persistent in-situ production of antimicrobials within a coaggregate or biofilm community, thus eliminating the need for continual dosing of a antimicrobial solution, which traditionally have needed to be at high concentrations to penetrate the biofilm.

In some embodiments, L. gummosus is engineered. For example, in some embodiments, L. gummosus is engineered to alter specificity or potency of killing. In some embodiments, L. gummosus is engineered to alter growth conditions (e.g., by adding genes for resistance to antibiotics or media conditions). Techniques for engineered bacteria are available.

The compositions comprising L. gummosus described herein find use in the killing or inhibition of growth of a variety of microorganisms (e.g., pathogenic bacteria or fungi).

Examples include but are not limited to, of Staphylococcys warneri, Staphylococcus epidermidis, Blastomonas natatoria, Methylobacterium sp., Sphingomonas sp., Streptococcus gordonii, Staphylococcus aureus 483, Streptococcus sp., Salmonella enterica subsp. Enterica, Candida albicans, Acinetobacter baumanii, Micrococcus luteus, Emticicia sp., Staphylococcus aureus, Chrysobacter sp., Staphylococcus capreae, Staphylococcus warneri, and Corynebacterium singular or Saccharomyces. In some embodiments, L. gummosus or compositions comprising L. gummosus find use in the treatment of bacterial infections in or on the body (e.g., bacterial infections in coaggregates or biofilms). In some embodiments, L. gummosus or compositions thereof are used to treat bacterial infections in wounds, sepsis, pathogenic bacterial infections in the stomach or intestine, and the like.

In some embodiments, pharmaceutical compositions are administering in a maintenance or ongoing manner (e.g., one or more times a day, two or more times a day, one or more times a week, etc.). In some embodiments, compositions are administered continuously (e.g., via a skin patch, bandage, or time release formulation). In some embodiments, compositions are administered once, twice, 5 times, 10 times or more. In some embodiments, compositions are administered over a period of weeks, months, years or indefinitely

In some embodiments, L. gummosus or compositions comprising L. gummosus find use in the decontamination of medical devices (e.g., catheters, speculums, and the like) or implantable medical devices (e.g., pacemakers, internal defibrillators, artificial joints or bones and the like).

In some embodiments, L. gummosus or compositions comprising L. gummosus find use in the decontamination of surfaces (e.g., surfaces comprising biofilms). Examples include but are not limited to, household surfaces, hospital or clinical surfaces (e.g., exam tables, operating rooms, etc.), and the like.

In some embodiments, L. gummosus or compositions comprising L. gummosus find use in the decontamination or protection of food or food preparation areas. For example, in some embodiments, L. gummosus is applied to a food after harvest to protect against future contamination or treat existing contamination.

### EXPERIMENTAL

The following examples are provided in order to demonstrate and further illustrate certain preferred embodiments and aspects of the present invention and are not to be construed as limiting the scope thereof.

### Example 1

### Materials and Methods

Sample Collection and Bacterial Culturing

Three showerheads were chosen for study. All three were located within domestic residences that were ˜2.5-10 miles apart. One domestic residence (showerhead UH) received well water while the other two residences (showerheads AH and HM) were geographically closer to one another and received water from the same metropolitan water supply. Showerhead biofilm samples were collected from the three domestic showerheads using BBL™ CultureSwab™ swabs (BD, Franklin Lakes, N.J.). These swabs were wetted in autoclaved filter sterilized tap water and approximately 5cm² of the outer surface of the showerhead was sampled. The swabs were suspended in 1.5 ml of a sterile 50% v/v glycerol solution. Tubes were agitated for 10 seconds to re-suspend the biofilm bacteria and then stored at −80° C.

For culturing, suspended biofilm samples were serially diluted in sterile distilled water to extinction. Dilutions were plated on R2A (Reasoner & Geldreich, Appl Environ Microbiol 49, 1-7, 1985) agar and incubated for 72 hours at 30° C. Colonies were differentiated based on colony color, size, and morphology. Only R2A agar was used in order to reduce the likelihood of re-isolating the same species that displayed culture-medium dependent colony morphologies. Dominant species were isolated and re-plated on solid R2A media, incubated for 72 hours at 30° C., and stored at 4° C. For long-term storage, strains were stored at −80° C. in 50% v/v glycerol solution.

In addition to those isolates derived from the showerhead biofilms (described here), the ability of L. gummosus HM010 to lyse target species included: Salmonella enterica subsp. enterica ATCC 14028, Acinetobacter baumanii ATCC 19606, and some strains described in Rickard et al. J Appl Microbiol. 108, 1509-1522 (2010), Rickard et al. Appl Environ Microbiol 68, 3644-3650 (2002), and Cisar et al. Infect Immun 24, 742-752 (1979). In addition, the susceptibility of Candida albicans ATCC MYA-2876 was tested.

### Bacterial Isolate Identification via Partial 16S rRNA PCR Amplification and Sequencing

For 16S rRNA sequencing, single colonies were suspended in 100 μl of PCR certified water (Promega, Madison, Wis.) and heated to 85° C. for 7 minutes. The suspension was centrifuged for 10 seconds at 6,400 RPM and 5 μl of each suspension was used as the template for PCR amplification of the 16S rRNA gene. PCR was performed using a mix of 25 μl GoTaq® Green Master Mix (Promega, Madison, Wis.), 18 μl PCR certified water (Promega, Madison, Wis.), and 1 μl each of forward primer (8FPL, AGTTTGATCCTGGCTCAG; SEQ ID NO:1) and reverse primer (806R, GGACTACCAGGGTATCTAAT; SEQ ID NO:2). The amplification protocol was identical to that used by Rickard et. al. (Rickard et al., Appl Environ Microbiol 70, 7426-7435, 2004b) except that a TC-5000 Thermo Cycler (Techne, Burlington, N.J.) was used. PCR products were cleaned using the QIAquick PCR Purification System (Qiagen, Valencia, Calif.) according to the manufacturer's protocol. The purified DNA was checked for quantity and purity using a NanoDrop 2000c spectrophotometer (Thermo Scientific, Watham, Mass.). Sequencing was performed by the DNA Sequencing Core at the University of Michigan (Ann Arbor, Mich.) using Applied Biosystems 3730x1 DNA Analyzers (Applied Biosystmes, Carlsbad, Calif.), BigDyev3.1 chemistry (MCLAB, San Francisco, Calif.), and the protocols recommended by the manufacturer. Resulting partial 16s rRNA gene sequences were analyzed using CHROMAS (Technelysium Pty. Ltd., Australia) and compared to known sequences in the National Center for Biotechnology (NCBI) database using the Basic Local Alignment Search Tool (BLAST).

### Phylogenetic Analysis

CLUSTALX v. 2.1 (Larkin et al., 2007) was used to initially align the partial 16s rRNA gene sequences with closely related strains in the NCBI database. Sequences of 620 nucleotides in length were used for tree construction. Aligned sequences were analyzed using TREECON v. 1.3b (Van de Peer & De Wachter, Comput Appl Biosci 9, 177-182, 1993) using the Jukes and Cantor (Jukes & Cantor, Evolution of protein molecules. In Mammalian protein metabolism pp. 21-132. Edited by H. N. Munro. New York: Academic Press Inc. 1969) substitution model. The 16S rRNA gene sequence from Thermus thermophilus (EMBL accession no. AJ251638) was used as an out-group.

### Culture-Independent Analysis

For culture-independent analysis, via pyrosequencing, dry BBL™ CultureSwab™ (BD, Franklin Lakes, N.J.) swabs were used to sample the three showerheads selected for study at the same time as samples were taken for culture-dependent analyses. Swab tips were removed and placed in labeled sterile 2.0 ml vials. These biofilm samples were frozen at −80° C. until needed for pyrosequencing which relied upon the use of the primers 939F (TTGACGGGGGCCCGCAC) and 1492R (TACCTTGTTACGACTT) primers were used for pyrosequencing. Briefly, in order to prepare for FLX sequencing, the size and concentration of DNA fragments were determined by using DNA chips within a Bio-Rad Experion Automated Electrophoresis Station (Bio-Rad Laboratories, Hercules, Calif.) and a TBS-380 Fluorometer (Promega Corporation, Madison, Wis.). A 9.6×106 sample of double-stranded DNA molecules/μl with an average size of 625 by were mixed with 9.6 million DNA capture beads, and subsequently amplified by emulsion PCR. After bead recovery and enrichment, the bead-attached DNAs were denatured with NaOH, and sequencing primers were annealed. A two-region 454 sequencing run was performed on a 70×75 GS PicoTiterPlate using a Genome Sequencer FLX System (Roche, Nutley, N.J.). Following sequencing, all failed sequence reads, low quality sequence ends (Avg Q25), short reads <150 by (final mean length 412 bp) and tags and primers were removed. Sequence collections were then depleted of any non-bacterial sequences, sequences with ambiguous base calls, sequences with homopolymers >5 bp in length, and chimeras as has been described previously (Bailey et al., Infect Immun 78, 1509-1519, 2010; Bloomfield et al., Clin Exp Allergy 36, 402-425, 2006; Callaway et al., J Anim Sci , 2010; Capone et al., J Invest Dermatol., 2011; Handl et al., FEMS Microbiol Ecol, 2011; Ishak et al., Microb Ecol 61, 821-831, 2011; Pitta et al., Microb Ecol 59, 511-522, 2010). To determine the predicted identity of microorganisms in the remaining sequences, sequences were queried using BLASTn against a highly curated custom database of high quality 16s bacterial sequences derived and manually curated from NCBI. Using a NET analysis pipeline, the resulting BLASTn outputs were compiled and data reduction analysis as described previously (Bailey et al., Infect Immun 78, 1509-1519, 2010; Bloomfield et al., Clin Exp Allergy 36, 402-425, 2006; Callaway et al., J Anim Sci , 2010; Capone et al., J Invest Dermatol., 2011; Handl et al., FEMS Microbiol Ecol, 2011; Ishak et al., Microb Ecol 61, 821-831, 2011; Pitta et al., Microb Ecol 59, 511-522, 2010). Bacteria were classified at the closest well-characterized genus (Bailey et al., Infect Immun 78, 1509-1519, 2010; Bloomfield et al., Clin Exp Allergy 36, 402-425, 2006; Callaway et al., J Anim Sci , 2010; Capone et al., J Invest Dermatol., 2011; Handl et al., FEMS Microbiol Ecol, 2011; Ishak et al., Microb Ecol 61, 821-831, 2011; Pitta et al., Microb Ecol 59, 511-522, 2010).

### Whole-Cell Hydrophobicity Assays

The surface hydrophobicity of the isolates was determined by using a modified approach of measuring bacterial adhesion to hexadecane described by Rosenberg (Rosenberg, Appl Environ Microbiol 42, 375-377, 1981; Rosenberg & Rosenberg, J Bacteriol 148, 51-57, 1981). Isolates were cultured in R2A broth for 48 hours at 30° C. in a rotary shaker-incubator shaking at 225 rpm. Cells were subsequently washed three times in sterile tap water by centrifugation, normalized to an OD600 of 1.0, and mixed in equal volumes with hexadecane (Sigma, 99%). Suspensions were vortexed for 60 s, and the two phases were allowed to separate for 10 min. Whole-cell hydrophobicity was expressed as the percent reduction in optical density difference between the original OD600 of cells in tap water and the OD600 of the hexadecane treated suspension.

### Visual Aggregation Assays

For visual coaggregation assays, isolates were cultured in R2A broth for 48 hours at 30° C. in a rotary shaker-incubator shaking at 225 rpm. Cell suspensions were subsequently washed and mixed in pair-wise combinations according to the method of Rickard et al. (Rickard et al., Appl Environ Microbiol 68, 3644-3650, 2002). The extent of coaggregation was scored using the protocol described by Cisar et. al (Cisar et al., Infect Immun 24, 742-75, 1979). Specifically, coaggregation between isolates was scored from 0 to 4 where: 0—no visible coaggregation in cell suspension; 1—small uniform coaggregates in suspension; 2—large coaggregates but suspension remains turbid; 3—large coaggregates which settle rapidly leaving some turbidity in the supernatant; 4—large coaggregates that settle immediately, leaving a clear supernatant. The cell suspensions containing single isolates were scored by the same manner to determine autoaggregation (self-aggregation) according to the method of Rickard et al (Rickard et al., 2003b, supra). Autoaggregation was scored by using the same criteria as those used for coaggregation. When autoaggregation occurred, the visual score assigned to the pair was determined by observing the relative drop in mixture turbidity and relative increase in aggregate flock size.

In order to gauge the ability of each isolate to coaggregate, each isolate was assigned a composite score based on the number of partners and relative strength of those individual coaggregations. The resulting score was referred to as the coaggregation index (CI). This score was calculated using the following equation:

CI=4(n₄)+3(n₃)+2(n₂)+(n₁)

Where:

CI=Coaggregation index of an isolate

nₓ=The number of coaggregations displayed by that isolate (n) with a visual coaggregation score of x. The visual coaggregation score (x) can be 4, 3, 2, or 1.

### Microscopy and Imaging

Coaggregates were visualized using a Leica Microsystems TCS SPE confocal laser scanning microscope (Leica, Exon, Pa.) and the LAS-AF acquisition software (Leica, Exon, Pa.). Isolates were grown under the same conditions as described for coaggregation assays. After growth, cultures were centrifuged for 4 minutes at 9,000×g, and washed 3 times with sterilized distilled water. Strains were stained with either 3.34 μM SYTO® 9 or 5.0 μM SYTO® 59 according to manufacturer protocols (Invitrogen, Carlsbad, Calif.). Equal volumes of stained cells were then combined in a glass culture tubes and agitated to allow for coaggregation. Following approximately 30 s of gentle agitation, 100 μl of the coaggregated pair was transferred to a glass microscope slide (VWR, Radnor, Pa.). To reduce disintegration of coaggregates and alteration of coaggregate structure, wells were created on microscope slides by layering 4 layers of parafilm M™ (Marathon, Menasha, Wis.) on the glass surfaces and excising a 10 mm×10 mm square with a razorblade. Pair-wise combinations with high >2 coaggregation scores were studied. Excitation and emission wavelengths were selected in accordance with manufacturer specifications for the fluorescent stains (Invitrogen, Carlsbad, Calif.). The resulting images were visualized and inspected in 3-dimensions using IMARIS (Bitplane, Zurich, Switzerland). Captured renderings were assembled in CORELDRAW v. X4 (Corel, Mountain View, Calif.).

### Plate (Colony) Biofilm Imaging: Spatio-Temporal Killing of Targeted Species by L. gummosus

Colony biofilms of L. gummosus HM010 were co-streaked on R2A agar with other species of bacteria and grown for up to eleven days. In order to examine the spatio-temporal nature of the killing, inoculation was performed by making “M” like streaks of each tested pair (L. gummosus and other species), where the tops of each “M”-streaked organism were approximately 1 cm apart. This procedure allowed for the critical inhibitory distance of each test microorganism to be evaluated and also, over time, allowed the L. gummosus to spread across the plate and decrease the effective distance between itself and the test organism (ultimately leading to the formation of colonies containing coaggregated mixtures of L. gummosus HM010 and the test organism). Plates were incubated at approximately 25° C. and video images were captured using a high resolution webcam.

### RESULTS

### Culture-Independent Analysis

The three showerhead biofilms were examined to determine their bacterial composition using bTEFAP FLX massively parallel pyrosequencing. Each showerhead biofilm possessed a unique bacterial community at the genus level (Table 1). The most dominant bacterial genera within the showerhead biofilms were members of the Xanthomonas, Methylobacterium, Lysobacter, Brevundimonas, and Flavobacterium. Each showerhead biofilm also possessed genera that were unique to that given showerhead (e.g. Enhydrobacter was present at 13.1% in showerhead AH but absent in both other showerheads). Genus-level differences extended to the level of bacterial class and upon averaging across the three showerhead biofilms, 50.3% of the sequences derived from bTEFAP were members of the gamma-proteobacteria (HM, 82.0%; AH, 28.0%; UH, 41.06%), 28.0% were members of the alpha-proteobacteria (HM, 11.4%; AH, 64.4%; UH, 8.3%), 7.53% were flavobacteria (HM, 1.3%; AH, 2.0%; UH, 19.3%), 5.1% were beta-proteobacteria (HM, 2.1%; AH, 0.2%; UH, 12.9%), 4.0% were bacilli (HM, 0.0%; AH, 0.1%; UH, 11.8%), and 2.4% were actinobacteria (HM, 1.8%; AH, 5.0%; UH, 1.2%). The remaining 2.7% comprised 10 other bacterial classes. DNA sequences from opportunistic pathogens, e.g. Staphylococcus and Pseudomonas, were detected in the showerhead biofilms (Table 1). Legionella sequences and Mycobacterium sequences were detected in the showerhead biofilm HM, but only at 0.1% and 0.2%, respectively (data not presented).

### Culture-Dependent Analysis

Culturing and plate counts on R2A agar indicated that all three showerheads possessed biofilms, albeit at greatly different culturable densities. Showerhead biofilm HM contained 1.70×10⁷ cfu/ml, showerhead AH contained 2.35×105 cfu/ml, and showerhead UH contained 1.15×10² cfu/ml.

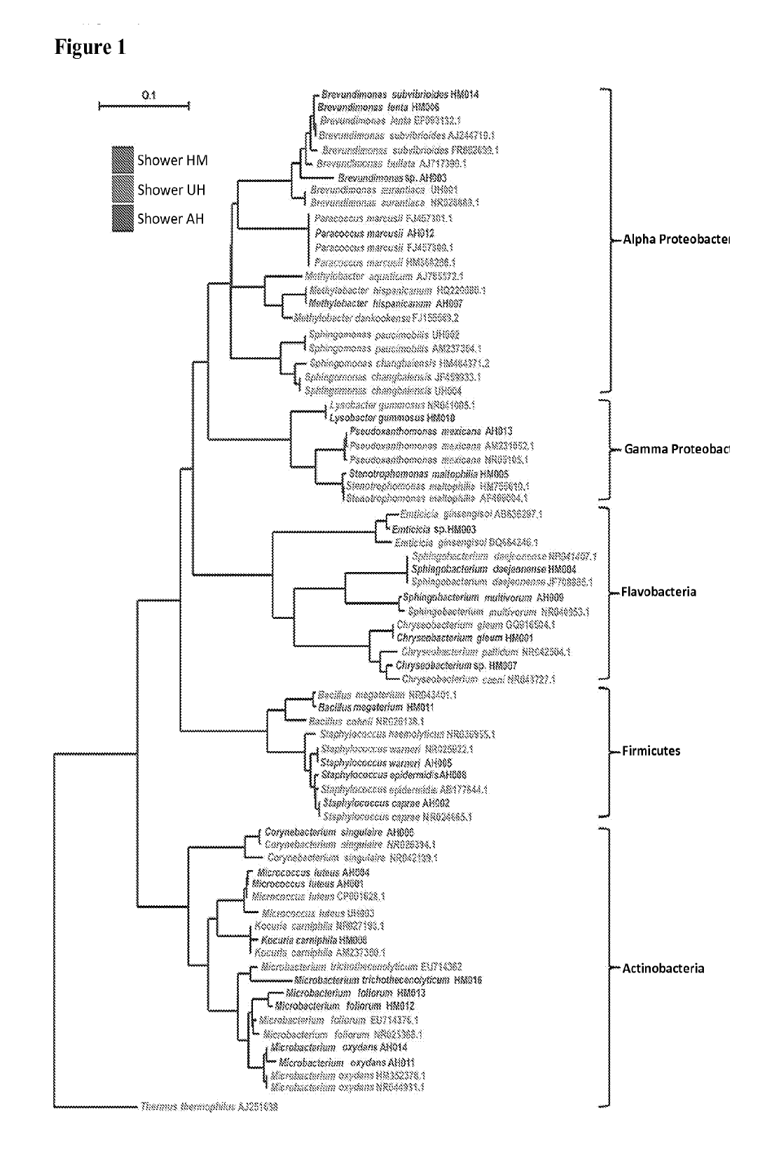

A total of 30 bacteria were isolated from the showerhead biofilms. Four isolates were cultured from showerhead UH, which received water from a potable well. Thirteen isolates each were isolated from showerheads AH and HM, which received water from the metropolitan water system. Isolates were identified as being from four phyla: the Actinobacteria, Firmicutes, Flavobacteria, and the Proteobacteria (alpha and gamma) (FIG. 1). Isolates belonging to the phyla Firmicutes and Flavobacteria as well as members of the gamma-Proteobacteria were exclusively isolated from the two showerheads that were fed from the metropolitan water system (AH and HM). Isolates belonging to the alpha-Proteobacteria and the Actinobacteria contained isolates from both showers. Only the genus Brevundimonas contained isolates from all three showerheads (FIG. 1). Twenty-seven of the 30 isolates were identified as having >97% identity to published bacterial 16S rRNA gene sequences (Table 2). These were consequently assigned species epithets.

### Autoaggregation and Whole-Cell Hydrophobicity

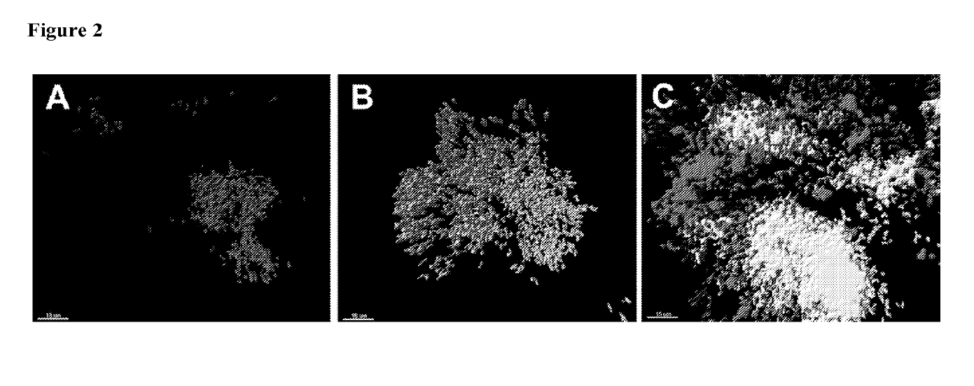

The propensity for a bacterium to autoaggregate may be related to whole-cell hydrophobicity and/or coaggregation ability. Autoaggregation ability was isolate-dependent and 13/30 of the isolates gave a positive visual autoaggregation score >1 (Table 3). The strongest autoaggregating isolates were Brevundimonas sp. AH003 (4), Corynebacterium singulare AH006 (4), Microbacterium trichothecenolyticum HM016 (4), Microbacterium hispanicum AH007 (4), and Sphingomonas changbaiensis UH004 (4). No relationship between autoaggregation score and % whole-cell hydrophobicity was evident (Table 3). For example, Staphylococcus warneri AH005 generated a hydrophobicity of 35.7% but did not autoaggregate while Microbacterium trichothecenolyticum HM016 generated a hydrophobicity of 39.9% and autoaggregated (visual score=4). Similarly, C. singulare AH006 generated a hydrophobicity of 5.6% and autoaggregated (visual score=4) while another isolate with similar low percentage hydrophobicity, Emticicia sp. HM003 (26.1%), did not autoaggregate. While no link was observed to coaggregation ability, autoaggregation had the potential to mask coaggregation, and this was taken into account when performing coaggregation assays. An example of this phenomenon was evident between M. hispanicum AH007 (autoaggregation score of 4) and M. trichothecenolyticum HM016 (autoaggregation score of 4) which was assigned a coaggregation score of 4 in recognition of the visually strong coaggregation interactions between the autoaggregated flocs (FIG. 2).

### Coaggregation Interactions

Every isolate from the three showerhead biofilms coaggregated with at least one other isolate, although the majority of pairwise combinations demonstrated only weak visual coaggregation scores. Of all the 435 possible pairwise combinations used to determine coaggregation, 32.8% (143/435) of pairs coaggregated to give a visual score of 1, 2, 3, or 4. Of the coaggregating partnerships, 2.1% (3/143) coaggregated at a score of 4, 2.1% (3/143) coaggregated at a score of 3, 12.6% (18/143) coaggregated at a score of 2, and 83.2% of pairs (119/143) coaggregated at a score of 1. With the exception of one isolate, M. trichothecenolyticum HM016, all isolates coaggregated with other isolates from the same showerhead biofilm from which it was isolated (intra-biofilm coaggregation, table 3). Every isolate was able to coaggregate with at least one other isolate from a different showerhead biofilm (inter-biofilm coaggregation, Table 3). Coaggregation also occurred at the inter-generic level, for example between B. lenta HM006 and B. aurantinca UH001 to give a visual score of 1, and at the intra-generic level, for example between B. lenta HM006 and M. luteus AH004 to give a visual score of 3. Isolate promiscuity can be determined by three measures: (i) number of coaggregation partners, (ii) average visual coaggregation score per partner, (iii) as a function of both the number of partners and the visual coaggregation score of each partnership. Some isolates had a high visual coaggregation score, but few partners (M. trichothecenolyticum HM016), and other isolates had a large number of partners, but the resulting visual scores were relatively low (P. mexicana AH013). In order to categorize isolate promiscuity as a function of both the number of partners and the resulting visual score of those partnerships the coaggregation index (CI) was determined for each isolate. Based upon this index, the most promiscuous coaggregating isolates were Brevindimonas lenta HM006 (17 partners, CI=28), Micrococcus luteus AH004 (16 partners, CI=25), and Lysobacter gummosus HM010 (14 partners, CI=20) (Table 3).

### Microscopic Analysis of Coaggregates

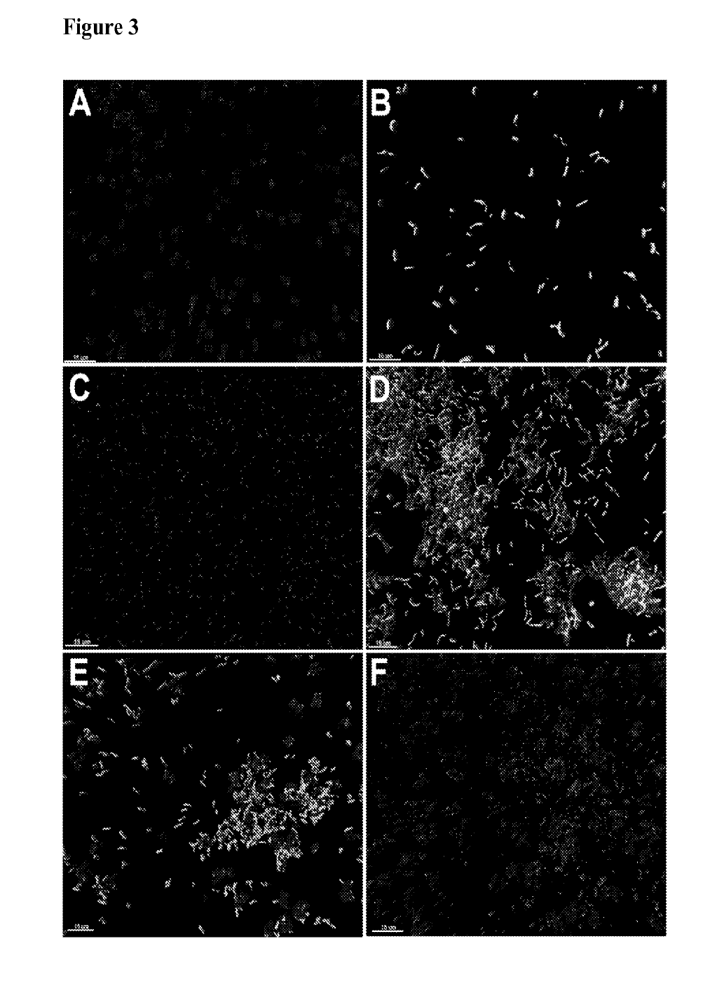

In order to discern structure, coaggregates that gave visual scores >2 were visualized using transmitted light microscopy and confocal scanning laser microscopy. Using confocal scanning laser microscopy in conjunction with different Syto® stains, single-isolate suspensions (FIG. 3A-C) and mixtures (coaggregated suspensions, FIG. 3D-F) were easily discerned. Microscopy of all coaggregates with a visual coaggregation of >2 showed that none of the coaggregate mixtures ever possessed (within or between samples) any defined architecture with respect to shape, size (from 2-3 cells to large coaggregated flocs >10004 in diameter) and spatial position of the isolates within the coaggregate. The visual strength of coaggregation clearly influenced the size of the coaggregate that was visualized by confocal scanning laser microscopy. The lower the visual score the smaller the coaggregate that were observed by microscopy and the more susceptible were the coaggregates to disassociation. For example, coaggregation between Brevundimonas lenta HM006 and Lysobacter gummosus HM010 (visual score of 4) and coaggregation between Micrococcus luteus AH004 and B lenta HM006 (visual score of 4) both yielded large densely-packed interdigitated coaggregated flocs with isolates wrapping around each other (FIG. 3D and E). This was in stark contrast to coaggregation between M. luteus AH004 and L. gummosus HM010 (visual score of 2) that, when visualized by confocal scanning laser microscopy, were mixtures of single-cells and small interdigitated coaggregates (FIG. 3F).

### Plate (Colony) Biofilm Imaging: Spatio-Temporal Killing by L. gummosus

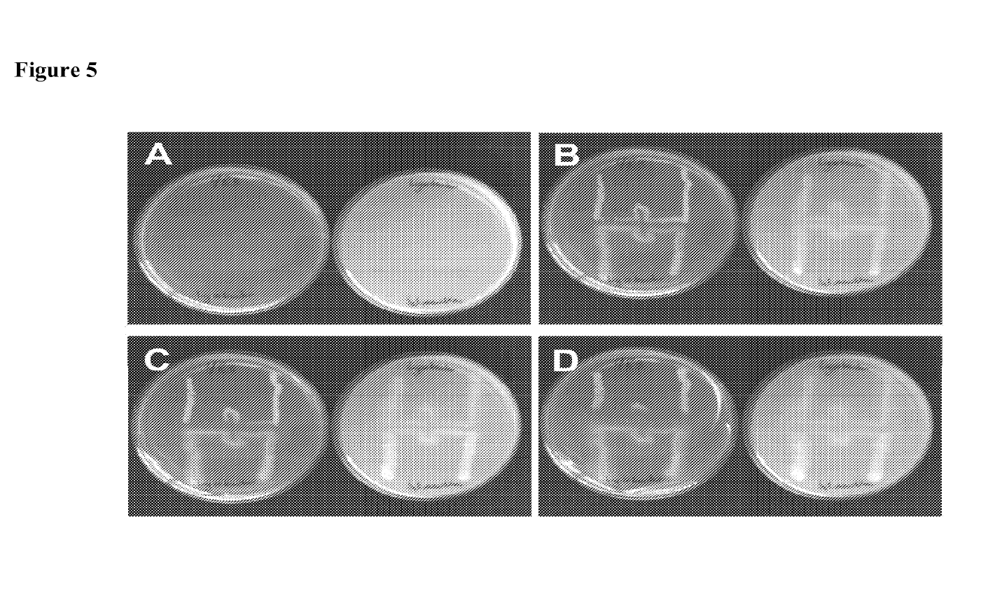

Camera images were collected over an eleven day period showing the co-growth of Lysobacter gummosus HM010 and variety of other species. An example of L. gummosus killing and lysing for a very susceptible species (S. aureus) and a less susceptible species (Salmonella enterica subsp. Enterica ATCC14028) is shown in FIG. 5. In FIG. 5, the clinically derived strain of Staphylococcus aureus is shown on the left plate and a culture-collection-derived strain of Salmonella enterica subsp. enterica is shown on the right plate. Both are subject to side-by-side inoculation of L. gummosus HM010. After initial inoculation of plates (time 0, FIG. 5A), killing (lysing) of S. aureus 483 already appeared as indicated by thinning-out of colony streaks that are close to L. gummosus HM010 (FIG. 5B). By even days incubation, extensive spreading and lysing of S. aureus 483 was observed, but little S. enterica ATCC14028 lysing was observed. By eleven days, vast sections of the colony biofilm of S. aureus 483 were lysed. In addition, some apparent lysing of S. enterica ATCC14028 was observed, although less pronounced than S. aureus 483. The present invention is not limited to a particular mechanism. Indeed, an understanding of the mechanism is not necessary to practice the present invention. Nonetheless, it is contemplated that the delayed lysing of S. enterica ATCC may be due to a need for juxtaposition of L. gummosus HM010 or a delayed release of an antimicrobial substance by L. gummosus HM010. Demonstrating the breadth of killing by L. gummosus HM010, other species demonstrated to be susceptible to lysis are listed in Table 4.

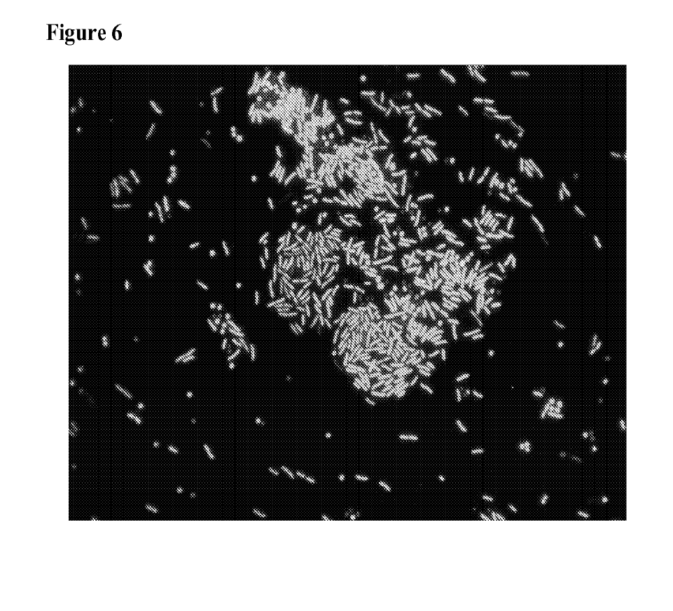

FIG. 6 shows an example of cell killing within a coaggregate. The epifluorescence micrograph shows a Live/Dead stained coaggregate containing L. gummosus HM010 and S. aureus CWS34 cells. Coccus-shaped cells (S. aureus CWS34 cells) are predominantly dead/damaged cells, especially within the coaggregate (center of image), while rod-shaped cells (L. gummosus HM010) are mostly live/viable cells.

TABLE 1
Estimated relative abundance of bacterial genera (%), as determined by bTEFAP,
of showerhead biofilms. The relative percentage of sequences assigned to a given
taxonomic classification (genera) for each individual showerhead sample are arranged
from highest to lowest across three showerheads. The average abundance across
the three showers is also shown. An asterisk (*) indicates that a member of this
genus was cultured from that showerhead biofilm (Table 2).

| | | Showerhead | Showerhead | Showerhead |
|---|---|---|---|---|
| Genus | Average | BiolfilmHM (%) | Biofilm AH (%) | BiofilmUH (%) |
| Xanthomonas | 18.67 | 37.46 | 3.92 | 14.60 |
| Methylobacterium | 12.58 | 3.98 | 33.77* | — |
| Lysobacter | 7.21 | 12.01* | 1.69 | 7.94 |
| Brevundimonas | 6.90 | 1.98* | 16.73* | —* |
| Flavobacterium | 6.87 | 1.30 | — | 19.31 |
| Psuedoxanthomonas | 6.86 | 13.55 | 2.59* | 4.44 |
| Methylophaga | 5.43 | 11.24 | 3.25 | 1.80 |
| Enhydrobacter | 4.35 | — | 13.06 | — |
| Curvibacter | 3.30 | — | — | 9.89 |
| Stella | 2.22 | — | — | 6.67 |
| Bacillus | 2.08 | —* | — | 6.24 |
| Staphylococcus | 1.83 | — | —* | 5.50 |
| Serratia | 1.50 | — | — | 4.50 |
| Sphingomonas | 1.38 | 1.02 | 3.13 | —* |
| Xylella | 1.12 | 2.72 | — | 0.63 |
| Enterobacter | 0.92 | — | — | 2.75 |
| Pseudomonas | 0.90 | 2.32 | 0.40 | — |
| Fulvimarina | 0.79 | — | 2.36 | — |
| Microbacterium | 0.75 | —* | 2.24* | — |
| Sphingobium | 0.68 | — | 2.03 | — |
| Chryseobacterium | 0.6 [symbol] | —* | 1.98 | — |
| Geobacter | 0.65 | — | — | 1.96 |
| Bdellovibrio | 0.63 | — | — | 1.90 |
| Moraxella | 0.51 | — | 1.54 | — |
| Brevibacterium | 0.49 | — | 1.46 | — |
| Photobacterium | 0.46 | — | — | 1.38 |
| Caulobacter | 0.42 | — | 1.26 | — |
| Aeromonas | 0.41 | — | — | 1.22 |
| Methylophilus | 0.35 | — | — | 1.06 |
| Rhodoferax | 0.35 | — | — | 1.06 |
| Micrococcus | 0.34 | — | 1.02* | —* |
| Propionibacterium | 0.32 | — | — | 0.95 |
| Rhizobium | 0.31 | — | 0.92 | — |
| Pedobacter | 0.23 | 0.68 | — | — |
| Phenylobacterium | 0.23 | 0.68 | — | — |
| Roseomonas | 0.23 | — | 0.68 | — |
| Dokdonelia | 0.21 | 0.62 | — | — |
| Blastochloris | 0.20 | — | 0.61 | — |
| Xenophilus | 0.20 | 0.59 | — | — |
| Acidobacterium | 0.19 | — | — | 0.58 |
| Afipia | 0.15 | 0.49 | — | — |
| Stenotrophomonas | 0.15 | 0.46* | — | — |
| Paracoccus | 0.13 | 0.40 | —* | — |
| Paracraurococcus | 0.13 | 0.40 | — | — |
| Methyloversatitis | 0.12 | 0.37 | — | — |
| Novosphingobium | 0.12 | 0.37 | — | — |
| Other Spp. | 5.44 | 7.35 | 3.37 | 5.61 |

[symbol]

indicates data missing or illegible when filed

TABLE 2
Identification of the showerhead biofilm isolates by alignment with 16S rRNA gene sequences
of published bacterial species in the NCBI database. An asterisk (*) indicates that
this genus was not identified through culture-independent techniques (Table 1).

| | | Closest Published | | | |
|---|---|---|---|---|---|
| | Sequence | Database | | | |
| Isolate | Length (b.p.) | Accession | Proposed Identity | % Identity | Accession Number |
| Showerhead | | | | | |
| Biofilm HM | | | | | |
| HM001 | 727 | AM159535.1 | Cryseobacterium gleum | 100 | HE800565 |
| HM003 | 714 | AB636297.1 | Emticicia sp. | 96 | HE800566 |
| HM004 | 744 | NR041407.1 | Sphingobacterrium daejeonense | 99 | HE800567 |
| HM005 | 742 | HM755619.1 | Stenotrophomonas matiophilia | 99 | HE800568 |
| HM006 | 681 | EF093132.1 | Brevundimonas lenta | 99 | HE800569 |
| HM007 | 739 | NR043727.1 | Chryseobacterium sp. | 96 | HE800570 |
| HM008 | 727 | NR027193.1 | Kocuria carniphila* | 98 | HE800571 |
| HM010 | 754 | NR041005.1 | Lysobacter gummosus | 99 | HE800572 |
| HM011 | 749 | FJ380122.1 | Bacillus megaterium | 99 | HE800573 |
| HM012 | 722 | HM233981.1 | Microbacterium folionum | 99 | HE800574 |
| HM013 | 732 | EU714376.1 | Microbacterium folionum | 99 | HE800575 |
| HM014 | 658 | EF093132.1 | Brevundimonsa subvibnoides | 99 | HE800576 |
| HM016 | 628 | EU714362 | Microbacterium trichothecenolyticum | 99 | HE800577 |
| Showerhead | | | | | |
| Biofilm AH | | | | | |
| AH001 | 702 | HM584233.1 | Micrococcus luteus | 99 | HE800578 |
| AH002 | 693 | JN644490.1 | Staphylococcus caprae | 99 | HE800579 |
| AH003 | 668 | AJ717390.1 | Brevundimonas sp. | 95 | HE800580 |
| AH004 | 691 | HM584251.1 | Micrococcus luteus | 99 | HE800581 |
| AH005 | 749 | JN644590.1 | Staphylococcus warneri | 99 | HE800582 |
| AH006 | 628 | NR026394.1 | Cornyebacterium singulare * | 99 | HE800583 |
| AH007 | 689 | HQ220080.1 | Methylobacterium hispanicum | 99 | HE800584 |
| AH008 | 740 | GU797281.1 | Stapylococcus epidermidis | 100 | HE800585 |
| AH009 | 754 | FJ548749.1 | Sphingobacterium multivorum* | 99 | HE800586 |
| AH011 | 692 | HM352379.1 | Microbacterium oxydans | 98 | HE800587 |
| AH012 | 687 | FJ457300.1 | Paracoccus marcusii | 99 | HE800588 |
| AH013 | 738 | FM213381.2 | Pseudooxanthomonas Mexicana | 99 | HE800589 |
| AH014 | 672 | JN613481.1 | Microbacterium oxydans | 100 | HE800590 |
| Showerhead | | | | | |
| Biofilm UH | | | | | |
| UH001 | 689 | GU204962.1 | Brevundimonas aurantiaca | 100 | HE800591 |
| UH002 | 679 | AY749436.1 | Sphingomonas paucimotilis | 99 | HE800592 |
| UH003 | 726 | CP001628.1 | Micrococcus luteus | 99 | HE800593 |
| UH004 | 686 | JF459933.1 | Sphingomonas changbaiensis | 99 | HE800594 |

TABLE 3
Autoaggregation, whole-cell hydrophobicity, and coaggregation partnerships between isolates from within the same shower
biofilm (intra-biofilm coaggregation) and with isolates from different shower biofilms (inter-biofilm coaggregation).
The isolates from each showerhead biofilm are arranged by coaggregation index (CI), from high to low CI.

| | | | | | Visual | | | | |
|---|---|---|---|---|---|---|---|---|---|
| | Numbers of | Numbers of | | | Coaggregation | | | | |
| | Intra-Biofilm | Inter-Biofilm | Autoaggregation | % Whole-Cell | Scores | | | | |
| Showerhead biolfilm/Isolate | Coaggregations | Coaggregations | Score | Hydrophobicity | 4 | 3 | 2 | 1 | CI* |
| Showerhead Biofilm HM | | | | | | | | | |
| B. lenta HM006 | 6 | 11 | 0 | 17.1 | 1 | 1 | 6 | 9 | 28 |
| L. gummosus HM010 | 6 | 8 | 0 | 4.9 | 1 | 1 | 1 | 11 | 20 |
| M. foliorum HM013 | 7 | [symbol] | 3 | 38.9 | 0 | 1 | 3 | 9 | 18 |
| C. gleum HM001 | 8 | 5 | 0 | 7.0 | 0 | 0 | 3 | 10 | 16 |
| K. carniphila HM008 | 4 | 7 | 0 | 0.6 | 0 | 0 | 2 | 9 | 13 |
| B. megaterium HM011 | 5 | 8 | 2 | 3.0 | 0 | 0 | 1 | 10 | 12 |
| M. foliorum HM012 | 4 | 6 | 3 | 29.4 | 0 | 0 | 2 | 8 | 12 |
| B. subvibrioides HM014 | 3 | 7 | 2 | 26.1 | 0 | 0 | 1 | 9 | 11 |
| S. maltophila HM005 | 2 | 7 | 0 | 3.9 | 0 | 0 | 1 | 8 | 10 |
| S. daejeonense HM004 | 4 | 5 | 0 | 4.8 | 0 | 0 | 0 | 9 | 9 |
| Emticicia sp HM003 | 2 | 6 | 0 | 26.1 | 0 | 0 | 0 | 8 | 8 |
| Chryseobacterium sp. HM007 | 5 | 1 | 0 | 4.9 | 0 | 1 | 0 | 5 | 8 |
| M. trichothecenolyticum HM016 | 0 | 2 | 4 | 39.9 | 1 | 0 | 1 | 0 | 6 |
| Showerhead Biofilm AH | | | | | | | | | |
| M. luteus AH004 | 5 | 12 | 1 | 14.0 | 0 | 2 | 4 | 11 | 25 |
| M. luteus AH001 | 6 | 9 | 1 | 2.9 | 0 | 0 | 1 | 14 | 16 |
| Brevundimonas sp. AH003 | 5 | 3 | 4 | 9.7 | 1 | 0 | 2 | 5 | 16 |
| M. hispanicum AH007 | 1 | 7 | 4 | 7.8 | 2 | 0 | 0 | 6 | 14 |
| P. mexicana AH013 | 3 | 8 | 0 | 3.0 | 0 | 0 | 1 | 10 | 12 |
| S. warneri AH005 | 5 | 5 | 0 | 35.7 | 0 | 0 | 1 | 9 | 11 |
| S. caprae AH002 | 2 | 6 | 3 | 10.4 | 0 | 0 | 2 | 6 | 10 |
| S. epidermidis AH008 | 2 | 6 | 0 | 11.0 | 0 | 0 | 0 | 8 | 8 |
| S. multivorum AH009 | 3 | 5 | 0 | 1.0 | 0 | 0 | 0 | 8 | 8 |
| C. singulare AH006 | 2 | 6 | 4 | 5.6 | 0 | 0 | 0 | 7 | 7 |
| M. oxydans AH014 | 3 | 4 | 0 | 13.6 | 0 | 0 | 0 | 7 | 7 |
| M. oxydans AH011 | 2 | 2 | 0 | 6.4 | 0 | 0 | 0 | 4 | 4 |
| P. marcusii AH012 | 1 | 2 | 1 | 9.8 | 0 | 0 | 0 | 3 | 3 |
| Showerhead Biofilm UH | | | | | | | | | |
| S. paucimobilis UH002 | 3 | 10 | 0 | 14.5 | 0 | 0 | 1 | 12 | 14 |
| B. aurantiaca UH001 | 2 | 7 | 0 | 9.8 | 0 | 0 | 1 | 8 | 10 |
| M. luteus UH003 | 3 | 6 | 0 | 11.5 | 0 | 0 | 1 | 8 | 10 |
| S. changbaiensis UH004 | 2 | 6 | 4 | 4.8 | 0 | 0 | 1 | 7 | 9 |

*= Coaggregation Index [symbol]
indicates data missing or illegible when filed

TABLE 4
Example of breadth of killing activity (lyric activity) of L. gummosus
HM010. Test strains were derived from multiple sources.

| Strain | Source | Reference |
|---|---|---|
| Emticicia sp. HM003 | Showerhead biofilm | This work |
| Chryseobacterium sp. | Showerhead biofilm | This work |
| HM007 | | |
| Micrococcus luteus AH001 | Showerhead biofilm | This work |
| Staphylococcus caprae | Showerhead biofilm | This work |
| AH001 | | |
| Micrococcus luteus AH002 | Showerhead biofilm | This work |
| Staphylococcys warneri | Showerhead biofilm | This work |
| AH005 | | |
| Staphylococcus epidermidis | Showerhead biofilm | This work |
| AH008 | | |
| Micrococcus luteus UH002 | Showerhead biofilm | This work |
| Blastomonas natatoria 2.4 | Freshwater biofilm | Rickard et al., 2002 |
| Methylobacterium sp. 2.7 | Freshwater biofilm | Rickard et al., 2002 |
| Micrococcus luteus 2.13 | Freshwater biofilm | Rickard et al., 2002 |
| Sphingomonas sp. 2.15 | Freshwater biofilm | Rickard et al., 2002 |
| Streptococcus gordonii DL1 | Dental plaque | Cisar et al. 1979 |
| Acinetobacter baumanii | Urine | ATCC 19606 |
| ATCC 19606 | | |
| Acinetobacter baumanii | Human chronic | Rickard et al. 2010 |
| CWS18 | wound | |
| Staphylococcus aureus 483 | Human chronic | Rickard et al. 2010 |
| (CWS34) | wound | |
| Salmonella enterica subsp. | Animal tissue | ATCC 1402 |
| enterica ATCC1402 | | |
| Candida albicans ATCC | Human clinical | ATCC MYA2876 |
| MYA2876 | specimen | |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of killing or preventing growth of a microorganism, comprising:
contacting bacteria with a composition comprising isolated Lysobacter gummosus, wherein said contacting kills or inhibits the growth of said microorganism.

2. The method of claim 1, wherein said microorganism is a bacteria.

3. The method of claim 2, wherein said bacteria are in a coaggregate.

4. The method of claim 3, wherein said Lysobacter gummosus coagreggates with said bacteria.

5. The method of claim 2, wherein said bacteria are present in a biofilm.

6. The method of claim 1, wherein said microorganisms are on a surface of an object.

7. The method of claim 6, wherein said object is a medical device.

8. The method of claim 1, wherein said microorganisms are in or on a subject.

9. The method of claim 8, wherein said microorganisms are in a wound.

10. The method of claim 2, wherein said bacteria are in a coaggregate or biofilm with a plurality of different bacterial species.

11. The method of claim 10, wherein said Lysobacter gummosus kills a subset of bacteria in said coaggregate or biofilm.

12. The method of claim 11, wherein said Lysobacter gummosus selectively kills specific bacterial species but not others.

13. The method of claim 1, wherein said Lysobacter gummosus selectively kills one or more microorganisms selected from the group consisting of Staphylococcys warneri, Staphylococcus epidermidis, Blastomonas natatoria, Methylobacterium sp., Sphingomonas sp., Streptococcus gordonii, Streptococcus sp., Staphylococcus aureus 483, Salmonella enterica subsp. Enterica, Candida albicans, Acinetobacter baumanii, Micrococcus luteus, Emticicia sp., Staphylococcus aureus, Chrysobacter sp., Staphylococcus capreae, Staphylococcus warneri, and Corynebacterium singular.

14. The method of claim 13, wherein said Staphylococcus aureus is an antibiotic resistant isolate.

15. The method of claim 1, wherein said Lysobacter gummosus is formulated as a pharmaceutical composition, a disinfecting, a cleaning solution, or is on a wound dressing.

16-28. (canceled)

29. A method of killing or preventing growth of bacteria in a biofilm, comprising:
contacting said biofilm with a composition comprising isolated Lysobacter gummosus, wherein said contacting kills or inhibits the growth of said bacteria in said biofilm.
